(19) United States
(12) Patent Application Publication
(10) Pub. No.: US 2005/0284474 A1
(43) Pub. Date: Dec. 29, 2005

(54) Nebulising medicine cup fixture assembly

(76) Inventor: Daniel C.M. Tseng, Taipei County (TW)

Correspondence Address: BIRCH STEWART KOLASCH & BIRCH, PO BOX 747, FALLS CHURCH, VA, 22040-0747, US

(21) Appl. No.: 10/875,268

(22) Filed: Jun. 25, 2004

(51) Int. Cl. A61M 15/00; A61M 16/10

(52) U.S. Cl. 128/203.16; 128/203.12

(57) ABSTRACT

A nebulising medicine cup fixture assembly simplifies the installation of a medicine cup to a medicine cup base of an air storage tank by redesigning the connection between the medicine cup and the medicine cup base of the air storage tank and assures the secure and airtight installation of the medicine cup. The connection measure taken by the invention comprises a protrusion and a recession having a fixing and latching relation with each other and being disposed at the positions corresponding to the medicine cup base of the air storage tank and the medicine cup, so that the medicine cup rotates with respect to the medicine cup base to latch the medicine cup into a fixed position by the protrusion and the recession, and finally the medicine cup can be fixed onto the air storage tank.

### FIELD OF THE INVENTION

The present invention relates to a nebulising medicine cup, more particularly to a simplified medicine cup installation method for assuring the secure installation of a medicine cup and the airtight nebulising medicine cup fixture assembly.

### BACKGROUND OF THE INVENTION

Please refer to a prior-art technology as disclosed in the R.O.C. Patent Publication No. 567855 and the U.S. patent Publication No. US2001/0002592 A1 for the structure of an apparatus for nebulising a liquid for medical use. Such apparatus nebulises a liquid medicine to maximize the effect of the medical treatment and the structure comprises a main body, an air compressor disposed in the main body, a nebulising device being interconnected with the air compressor and capable of containing a liquid medicine, and an output device. The air compressor rapidly compresses the air and sends the compressed air into the nebulising device, and then the nebulising device produces a nebulization effect onto the liquid medicine in the nebulising device, and finally the output device outputs the nebulised medicine for the medical treatment.

The foregoing prior-art technology makes use of a screw connection method to fix the medicine cup of the nebulising device onto the air storage tank of the air compressor; wherein the medicine cup at its bottom has a locking section corresponding to the threaded section of the air storage tank. When the locking section of the medicine cup is connected with the threaded section of the air storage tank by screwing, the compressed air is sent into the nebulising device to produce the nebulization effect.

However, when such prior-art technology is applied for installing the medicine cup onto the air storage tank, the airtight effect is poor after the medicine cup is installed because the locking section of the medicine cup cannot be aligned precisely with the threaded section of the air storage tank easily, or it is necessary to install the medicine cup onto the air storage tank several times to fix the locking section and the threaded section, and thus making the installation more complicated.

### SUMMARY OF THE INVENTION

The primary objective of the present invention is to overcome the foregoing shortcomings and avoid the existing deficiency by providing a simplified medicine cup installation method for assuring the secure installation of the medicine cup and the airtight nebulising medicine cup fixture assembly.

To achieve the foregoing objective, the nebulising medicine cup fixture assembly according to the present invention uses a connection measure and builds a protrusion and a recession having a fixing and latching relation with each other and being disposed at the positions corresponding to the medicine cup base of the air storage tank and the medicine cup, such that the medicine cup rotates with respect to the medicine cup base in order to latch the medicine cup into a fixed position by the protrusion and the recession, and finally the medicine cup can be fixed onto the air storage tank. Since the foregoing installation is simple and easy, therefore the time required for installing the medicine cup to the air storage tank is reduced and the present invention also assures a secure and airtight installation.

### BRIEF DESCRIPTION OF THE DRAWINGS

FIG. 1 is a perspective view of the present invention when it is in use.

FIG. 2 is a perspective view of the present invention.

FIG. 3 is an exploded view of the present invention.

FIG. 4A and 4B are side views of the motions of the present invention.

FIG. 5 is an exploded view of a first preferred embodiment of the present invention.

FIG. 6 is an exploded view of a second preferred embodiment of the present invention.

### DETAILED DESCRIPTION OF THE PREFERRED EMBODIMENTS

The detailed description and technical characteristics of the present invention are described together with the drawings as follows.

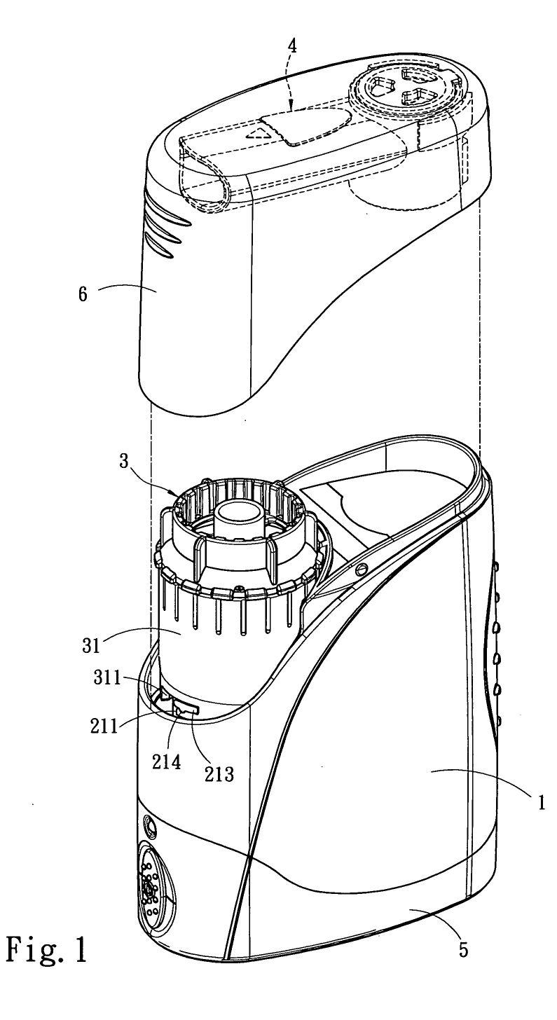

Please refer to FIG. 1 for the perspective view of the present invention when it is in use. The present invention as shown in the figure comprises a main body 1, an air compressor (not shown in the figure) being disposed in the main body 1, a nebulising device 3 being disposed in the air compressor and capable of containing a liquid medicine, a nozzle 4 installed on the nebulising device 3, a base 5 being installed at the bottom of the main body 1 for turning on the air compressor, and a protective cover 6 for covering the main body 1. The present invention uses the air compressor to compress the air rapidly and sends the compressed air into the nebulising device 3. The nebulising device 3 produces a nebulization effect to a liquid medicine contained in the nebulising device and then outputs the nebulised medicine through the nozzle 4 for medical treatments.

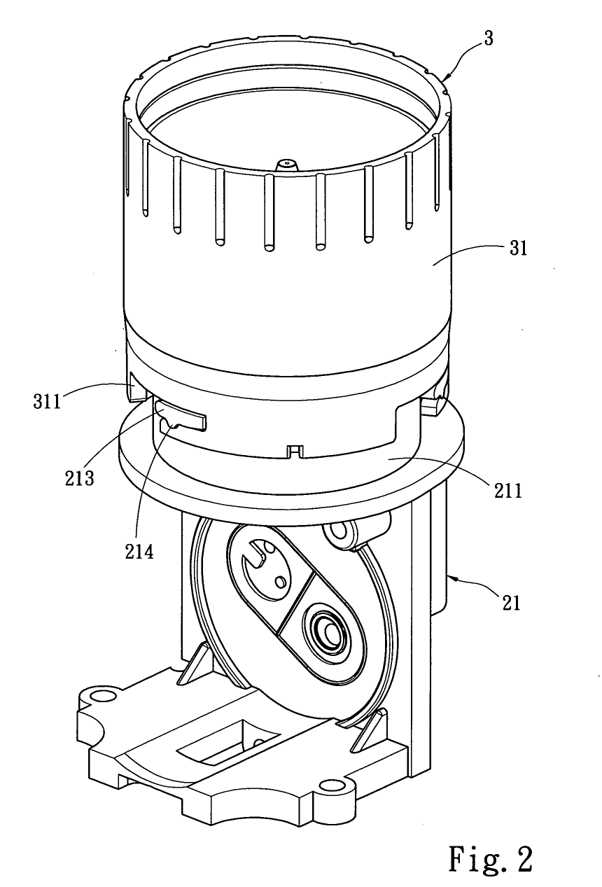

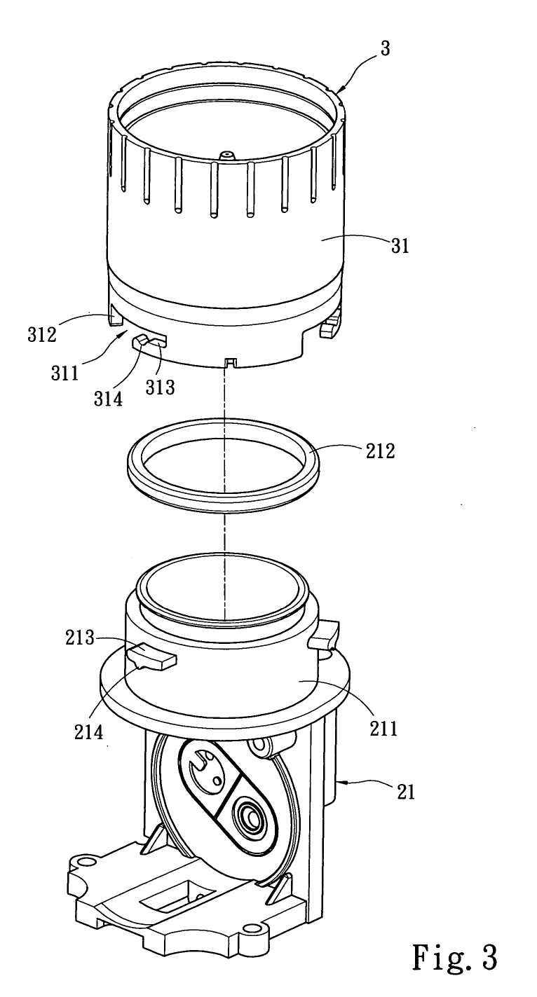

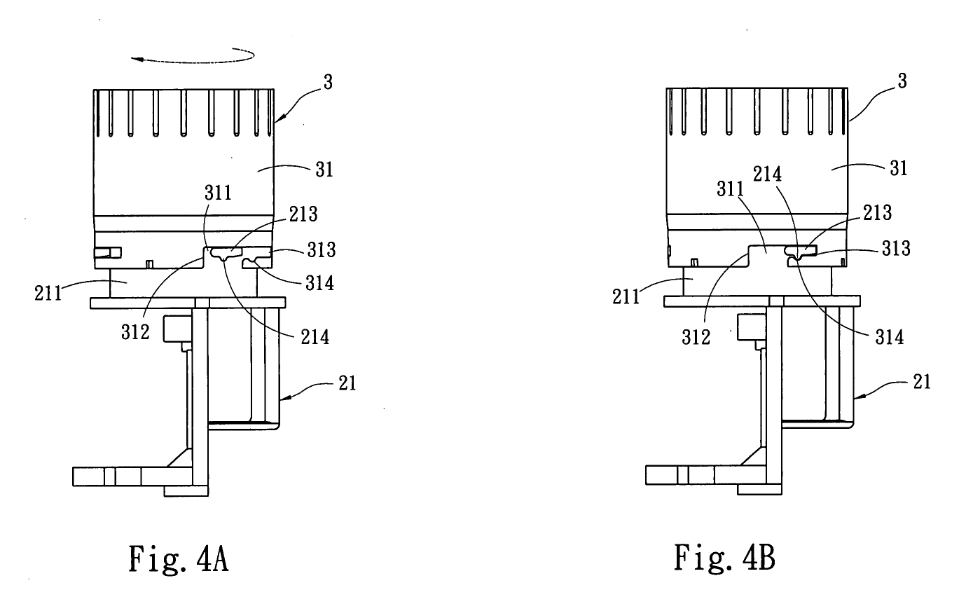

Please refer to FIGS. 2, 3, 4A and 4B respectively for the perspective view, exploded view and motions of the present invention. The present invention as shown in the figures comprises an air storage tank 21 of the air compressor, and the air storage tank 21 having a medicine cup base 211 for installing a medicine cup 31 of the nebulising device 3 and an airtight ring 212 sheathed into an opening of the medicine cup base 211 for preventing gas leakage; wherein the internal diameter of the foregoing medicine cup 31 is equal to the diameter of the medicine cup base 211 of the air storage tank 21; the medicine cup 31 has a plurality of grooves being disposed at the bottom of the medicine cup 31 and passing through the inner wall and outer wall of the medicine cup 31; the groove 311 has a connecting area 312 and a latch area 313; the latch area 313 has a recession 314; the medicine cup base 211 of the air storage tank 21 has a plurality of latch sections 213 disposed on an end surface corresponding to the groove 311; the latch section 213 has a protrusion 214 corresponding to the recession 314. The medicine cup 31 is installed onto the medicine cup base 211 of the air storage tank 21, such that the latch section 213 enters into the connecting area 312 of the groove 311 and rotates the medicine cup 31 to drive the latch section 213 to slide into the latch area 313 and latch the protrusion 214 of the latch section 213 into the recession 314 of the latch area 313 to enhance the strength of latching the medicine cup 31 to the medicine cup base 211 and prevent the medicine cup 31 to slide down easily and secure the medicine cup 31 onto the air storage tank 21.

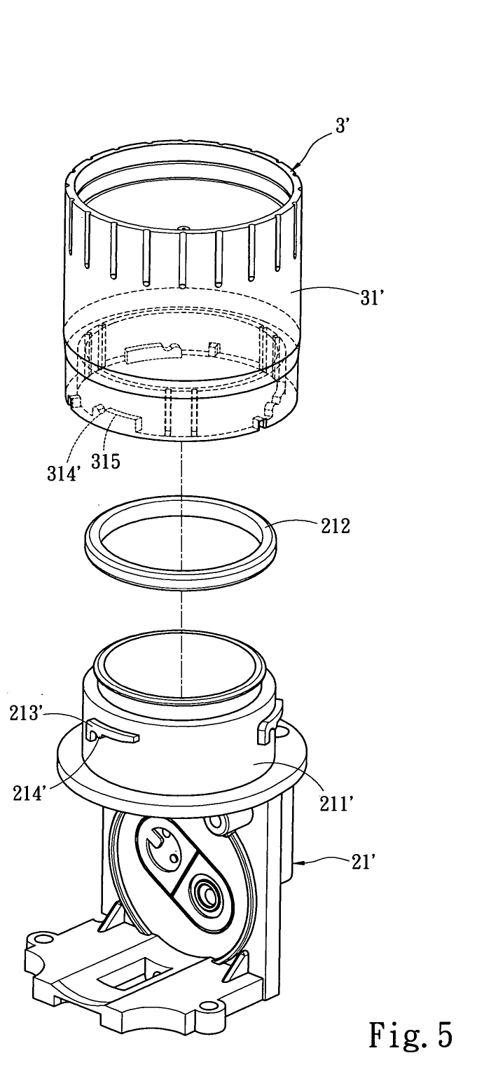

Please refer to FIG. 5 for a first preferred embodiment of the present invention. In the figure, the structure of this embodiment is substantially the same as the previous embodiment, and the difference resides on that the internal diameter of the medicine cup 31′ is slightly larger than the diameter of the medicine cup 211′ of the air storage tank 21′, and a protruded member 315 is disposed on the inner wall of the medicine cup 31′, and the protruded member 31 has a recession 314′. The medicine cup 31′ is installed onto the medicine cup 211′ of the air storage tank 21′, so that the latch section 213′ enters into the medicine cup 31′ and rotates the medicine cup 31′ to slide the latch section 213′ to touch the top of the protruded member 315 and the protrusion 214′ of the latch section 213′ latches the recession 314′ of the protruded member 315 to fix the medicine cup 31′ onto the air storage tank 21′.

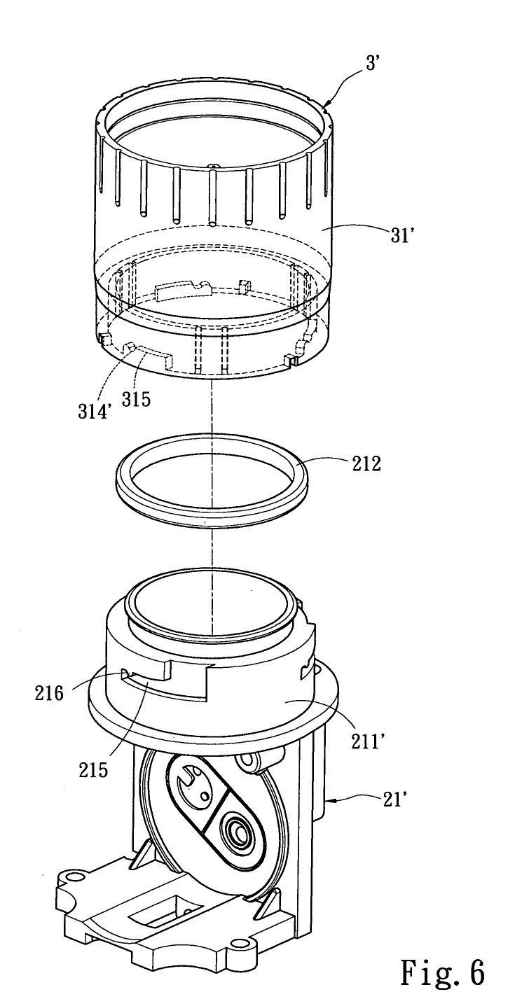

Please refer to FIG. 6 for a second preferred embodiment of the present invention. The difference between this embodiment with the first preferred embodiment resides on that the medicine cup base 211′ comprises a groove 215 disposed at a position corresponding to the protruded member 315 of the medicine cup 31′ and the groove 215 comprises a protrusion 216 to couple the medicine cup 31′ onto the medicine cup base 211′, so that the protruded member 315 enters into the groove 215 of the medicine cup base 211′. When the medicine cup 31′ is rotated, the recession 314′ of the protruded member 315 is latched to the protrusion 216 of the groove 215 in order to fix the medicine cup 31′ onto the air storage tank 21′.

1. A nebulising medicine cup fixture assembly, comprising: a main body;
an air compressor, being disposed in said main body and having an air storage tank and a medicine cup base being disposed on said air storage tank;
a nebulising device, being installed in said air compressor and having a medicine cup;
a nozzle, being coupled to said nebulising device;
wherein said medicine cup base of said air storage tank and said medicine cup define a protrusion and a recession having a fixing and latching relation at their corresponding positions, thereby said medicine cup rotates with respect to said medicine cup base of said air storage tank and is latched by said protrusion and said recession.

2. The nebulising medicine cup fixture assembly of claim 1, wherein said medicine cup has an internal diameter substantially equal to the diameter of said medicine cup base of said air storage tank and said connection measure is to set a plurality of grooves being disposed at the bottom of said medicine cup and passing through the inner and outer walls of said medicine cup, and said groove has a connecting area and a latch area, and said latch area has a recession and said medicine cup base of said air storage tank has a plurality of latch sections corresponding to the end surface of said groove, and said latch section has a protrusion corresponding to said recession; said medicine cup is disposed and rotated on the medicine cup base of said air storage tank such that said latch section enters into a connecting area of said groove and slides into said latch area and latches said protrusion of said latch section into said recession of said latch area to fix said medicine cup onto said air storage tank.

3. The nebulising medicine cup fixture assembly of claim 1, wherein said medicine cup has an internal diameter substantially equal to the diameter of said medicine cup base of said air storage tank and said connection measure is to set a plurality of protruded members on the inner wall of said medicine cup, and said protruded member has a recession; said medicine cup base of said air storage tank has a plurality of latch sections disposed at the end surface of said protruded member corresponding to said recession; said medicine cup is installed and rotated on said medicine cup base of said air storage tank such that said protrusion of said latch section latches with said recession of said protruded member to fix said medicine cup onto said air storage tank.

4. The nebulising medicine cup fixture assembly of claim 1, wherein said medicine cup base at its opening comprises an airtight ring disposed for preventing gas leakage.

5. The nebulising medicine cup fixture assembly of claim 1, wherein said medicine cup at its inner wall comprises a plurality of protruded members, and said protruded member has a recession, and said medicine cup base comprises a groove disposed at a position corresponding to said protruded member, and said groove comprises a protrusion, such that when said medicine cup is coupled and rotated on said medicine cup base, said recession of said protruded member is latched with said protrusion of said groove to fix said medicine cup onto said air storage tank.
